(19) United States
(12) Patent Application Publication
(10) Pub. No.: US 2025/0268548 A1
(43) Pub. Date: Aug. 28, 2025

(54) DISPLAY CONTROL APPARATUS, DISPLAY CONTROL METHOD, AND NON-TRANSITORY COMPUTER-READABLE RECORDING MEDIUM

(71) Applicant: KONICA MINOLTA, INC., Tokyo (JP)

(72) Inventor: Naoki HAYASHI, Tokyo (JP)

(73) Assignee: KONICA MINOLTA, INC., Tokyo (JP)

(21) Appl. No.: 19/058,314

(22) Filed: Feb. 20, 2025

(30) Foreign Application Priority Data
Feb. 22, 2024 (JP) 2024-025740

(51) Int. Cl. A61B 6/46

(52) U.S. Cl. CPC A61B 6/463; A61B 6/468

(57) ABSTRACT

A display control apparatus includes a hardware processor that sets a display order of the plurality of analysis results for a radiographic image to be displayed on a display, and the hardware processor performs control such that the plurality of analysis results are sequentially displayed on the display in a plurality of times in accordance with the set display order.

### CROSS REFERENCE TO RELATED APPLICATIONS

The entire disclosure of Japanese patent Application No.2024-025740, filed on Feb. 22, 2024, is incorporated herein by reference in its entirety.

### BACKGROUND

### Technological Field

The present invention relates to a display control apparatus, a display control method, and a non-transitory computer-readable recording medium.

### Description of Related Art

With the search for diagnostic utilization for a case using a radiographic image by radiography, various kinds of analysis processing have been developed, operated, and deployed, and the analysis processing has increased in various ways. Accordingly, since a plurality of types of analysis images are generated from one radiographic image, a technician who provides the radiographic image and a doctor who checks the radiographic image also needs to search for and check appropriate analysis processing from among a wide variety of analysis processing.

For example, Japanese Unexamined Patent Publication No. 2022-114195 discloses a configuration in which a priority is set for each disease of a subject with respect to each analysis result and a list of images based on the set priority is displayed. This configuration causes the analysis result of the image selected by the user from the list of images to be displayed.

### SUMMARY

However, in the configuration described in Japanese Unexamined Patent Publication No. 2022-114195, when each analysis result is displayed, an operation for the user to select an image corresponding to the type of analysis processing or the analysis result is required each time. In an actual clinical practice, a user may not have time to select an image, and the user needs to stop working for the above-described operation each time, which may be troublesome for the user. In particular, assuming that work in a medical site (e.g., interpretation of a radiographic image or clinical practice) is carried out as a routine, the above-described problem becomes more prominent.

An object of the present invention is to provide a display control apparatus, a display control method, and a non-transitory computer-readable recording medium capable of suppressing the occurrence of time and effort when a user checks display of a plurality of analysis results.

In order to achieve at least one of the above-described objects, a display control apparatus reflecting one aspect of the present invention includes:
a hardware processor that sets a display order of a plurality of analysis results for a radiographic image to be displayed on a display, in which
the hardware processor performs control such that the plurality of analysis results are sequentially displayed on the display in a plurality of times in accordance with the set display order.

In order to achieve at least one of the above-described objects, a display control method reflecting one aspect of the present invention is a method for controlling display of a plurality of analysis results for a radiographic image, and includes:
setting a display order of the plurality of analysis results on a display; and
performing control such that the plurality of analysis results are sequentially displayed on the display in a plurality of times in accordance with the set display order.

In order to realize at least one of the above-described objects, a non-transitory computer-readable recording medium reflecting one aspect of the present invention is a non-transitory computer-readable recording medium storing a display control program for a plurality of analysis results for a radiographic image, the display control program causing a computer to execute:
setting a display order of the plurality of analysis results to be displayed on a display; and
controlling such that the plurality of analysis results are sequentially displayed on the display in a plurality of times in accordance with the set display order.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a block diagram illustrating an image display system including a display control apparatus according to an embodiment of the present invention;

FIG. 2 is a block diagram illustrating the configuration of a main part of an image analysis apparatus;

FIG. 3 is a diagram illustrating an example of a screen transition of a display based on the display order;

FIG. 4 is a diagram illustrating an example of 2×1 display on the display;

FIG. 5 is a diagram illustrating an example of data in which a plurality of display order patterns are associated with combinations of elements of examination information and imaging information;

FIG. 6 is a flowchart illustrating an example of processing of a controller;

FIG. 7 is a diagram illustrating an example in which information on an image to be displayed next is displayed on the display; and

FIG. 8 is a diagram showing an example of a 2×2 display in the display.

### DETAILED DESCRIPTION OF EMBODIMENTS

Hereinafter, one or more embodiments of the present invention will be described with reference to the drawings. However, the scope of the invention is not limited to the disclosed embodiments.

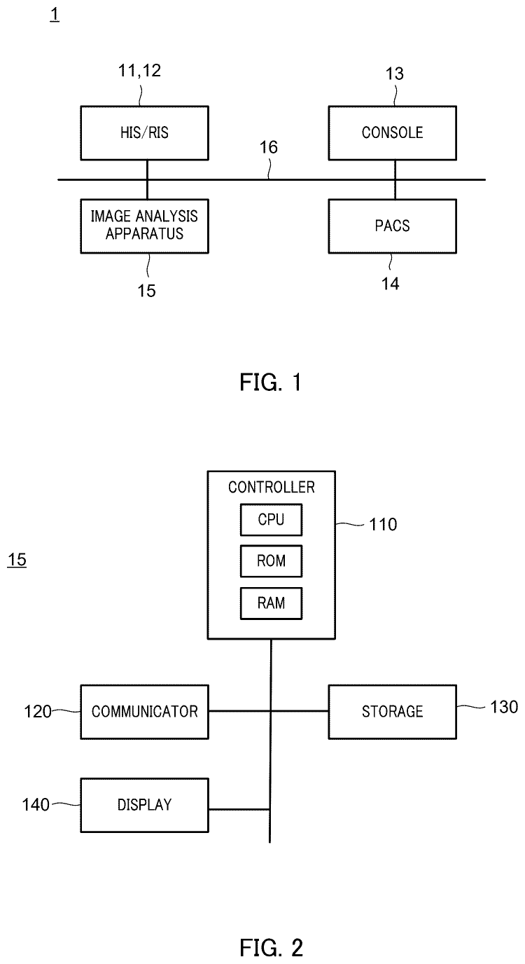

Hereinafter, an embodiment of the present invention will be described in detail with reference to the drawings. FIG. 1 is a block diagram which shows an image display system 1 including a display control apparatus according to an embodiment of the present invention.

As shown in FIG. 1, the image display system 1 is a system capable of displaying an image (radiographic image) generated by a modality (not shown). The image display system 1 includes a hospital information systems (HIS) 11, a radiology information systems (RIS) 12, a console 13, a picture archiving and communication systems (PACS) 14, and an image analysis apparatus 15. These are communicable with each other via a communication network 16.

Further, the modality is, for example, a radiographic image capturing apparatus, and may be an apparatus capable of generating a dynamic image constituted by a plurality of frame images or an apparatus capable of generating a still image. Note that the modality may be connected to the communication network 16 via the console 13. In addition, the transfer of the image from the modality to the image display system 1 may be performed via the console 13 or may be performed by using a storage medium or a cable.

The HIS11 and the RIS12 store examination information. The examination information may be, for example, the type of imaging (the type of modality to be used, distinction between a standing position and a lying position, and the like), an imaging target region, the name of a medical department that has requested the examination, the age of the subject, the sex of the subject, the name of the operator, and the like. Based on a request from another device (e.g., the console 13, the PACS14, or the image analysis apparatus 15), the HIS11 and the RIS12 transmit examination information corresponding to the request to the other device.

The console 13 is constituted by a personal computer (PC), a dedicated device, or the like. The console 13 sets various imaging conditions (a tube voltage, a tube current, an irradiation time (mAs value), a frame rate, and the like) for the modality and the like, based on examination information acquired from other apparatuses (the HIS11, the RIS12, and the like) or operation by the operator. The console 13 also performs various controls for starting and ending image capturing, acquisition of a captured image, and output of the captured image to the PACS14 or image analysis apparatus 15.

The PACS14 is constituted by a PC, a dedicated device, or the like. The image processing apparatus PACS14 includes a database, and stores data such as a still image or a dynamic image acquired from a modality in the database. In response to a request from another device (e.g., the console 13 or the image analysis apparatus 15), the PACS14 transmits, to the other device, an image or the like corresponding to the request from among images or the like stored in the database.

The image analysis apparatus 15 is a device capable of displaying an image based on an operation by an operator, such as a PC, a portable terminal (for example, a tablet terminal or a smartphone), or a dedicated device, and analyzes a still image or a dynamic image acquired from a modality via the console 13. The image analysis apparatus 15 corresponds to a “display control apparatus” of the present invention.

The image analysis apparatus 15 includes a controller 110, a communicator 120, a storage 130, and a display 140.

The controller 110 includes a central processing unit (CPU), a read only memory (ROM), a random access memory (RAM), and the like. The controller 110 causes the CPU to read a program corresponding to the processing details from the ROM and develop it in the RAM. Next, the controller 110 cooperates with the expanded program to centrally control the operation of each part.

The communicator 120 is constituted by a wireless module or the like, and transmits and receives various signals, and various types of information to and from other devices connected via the communication network 16.

The controller 110 acquires, via the communicator 120, image data of the radiographic image generated by the modality. After image data is acquired, the controller 110 analyzes the image data and generates an analysis image that is an analysis result of the image data.

The analysis images may include, for example, a blood flow analysis image, a structure analysis image, a ventilation analysis image, an adhesion analysis image, a diaphragm movement amount analysis image, and a shaping-related measurement image. In addition, the analysis images may include an abnormality detection image indicating a portion in which an abnormality is detected, an analysis summary image, a past comparison image indicating comparison with a past image, a past time difference image indicating a time difference from the past, and the like. Further, the analysis image may include an image that is not subjected to the analysis processing but is used for comparison with an image subjected to the analysis processing, such as an original image or a past image captured in the past. Furthermore, the analysis image may include an image based on analysis processing other than the above-described examples.

The controller 110 stores these analysis images in the storage 130.

These analysis images are checked by a user by being displayed on the display 140 after analysis by the controller 110. The user includes, for example, a doctor such as a radiologist or a clinician, an examination engineer of the radiographic image capturing apparatus, or an assistant of the doctor or the examination engineer.

In a case where a plurality of analysis results are stored as described above, a user needs to select an image to be checked from among the plurality of analysis results. In order for the user to perform the operation for the selection, the user has to stop the work being executed each time, which may be troublesome for the user.

Therefore, in the present embodiment, the controller 110 sets the display order of the plurality of analysis results for the radiographic image on the display 140. The controller 110 corresponds to a “setter” according to the present invention.

For example, the controller 110 may set the display order in accordance with a combination of elements included in the examination information, the imaging information, and the like.

The examination information includes, for example, information on a medical department related to the capturing of the radiographic image and information on the purpose of the examination. The medical department may be any medical department, for example, emergency, respiratory medicine, thoracic surgery, cardiovascular medicine, or the like, as long as the medical department uses radiographic images for diagnosis. The purpose of the examination may be any purpose such as screening, chronic obstructive pulmonary disease (COPD), pneumothorax, postoperative progress monitoring, or the like.

The imaging information includes information on an examination region of the subject, information on the presence or absence of a past examination, information on an imaging direction, information on the number of times of imaging, and the like. The examination region may be any region that can be examined using a radiographic image, such as the chest or the abdomen, for example. The imaging direction may be any direction, such as a front direction or a side direction, as long as a radiographic image can be captured.

For example, the following is assumed: a medical department which governs a subject to be radiographed is respiratory medicine, the purpose of examination is screening, and images of the abdomen have been captured from the front. In the image captured by the radiation imaging in this example, it is assumed that the controller 110 has generated an adhesion analysis image and a ventilation analysis image as analysis results for the original image.

In this case, for example, the controller 110 sets a display order in which the original image, the ventilation analysis image, and the adhesion analysis image are displayed in this order.

Thus, when the user checks the analysis result on the display 140, the analysis results are displayed on the display 140 in the order of the original image, the ventilation analysis image, and the adhesion analysis image.

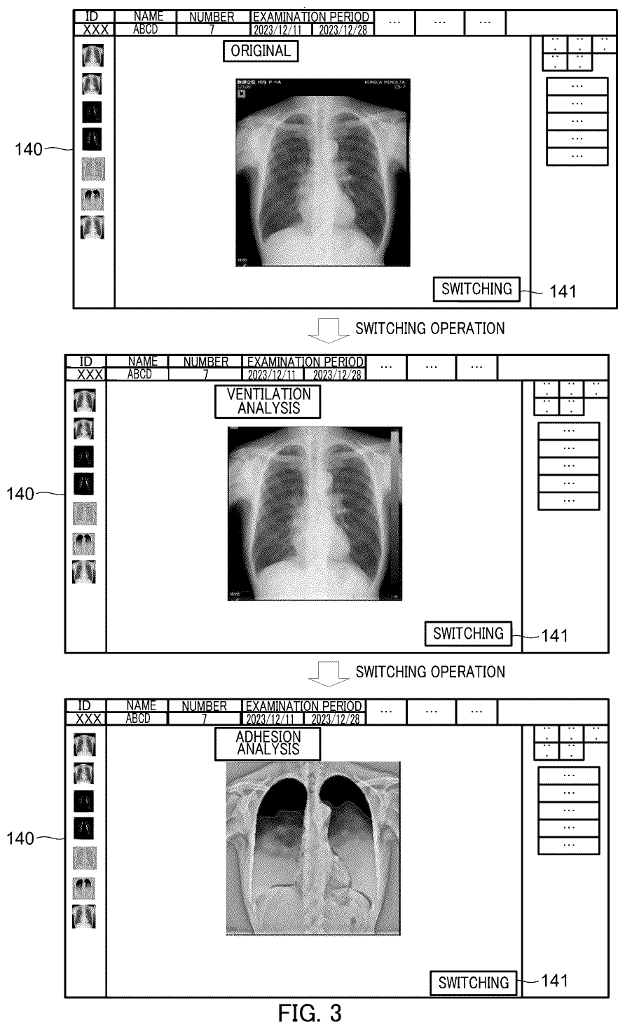

As illustrated in FIG. 3, the switching of images on the display 140 may be performed, for example, by a user operating a switching button 141 provided on the screen of the display 140. Specifically, when the user causes the display 140 to display the analysis result, first, the original image (“original” in FIG. 3) is displayed. When the user presses the switch button 141 to perform a switching operation, the display on the display 140 is switched from the original image to the ventilation analysis image (ventilation analysis in FIG. 3). Then, when the user performs a switching operation by pressing the switching button 141, the display of the display 140 is switched from the ventilation analysis image to the adhesion analysis image (adhesion analysis in FIG. 3). Herein, in FIGS. 3, 4, 7, and 8, “number” refers to “examination image number.”

This saves users the trouble of having to search through a plurality of analysis results to find the image the user wants to view.

Furthermore, switching of images on the display portion 140 may be automatically performed in accordance with the lapse of time, or may be performed by an operation other than the operation of the switching button 141 by the user. The operation other than the operation of the switching button 141 may be, for example, an operation of switching all images to be displayed N-th (any natural number) when there are a plurality of images to be displayed N-th. After this operation is performed, the (n+1)-th image is displayed on the display 140. Furthermore, the operation other than the operation of the switching button may be, for example, an operation of sliding an image within the screen of the display 140 in a case where the display 140 is of a touch panel type. Furthermore, when two types of analysis images are displayed, the switching of the images on the display 140 may be performed after all the frames or a specific frame range of the analysis image based on one of the dynamic images is displayed.

In addition, when a dynamic image is included in the analysis result, the operation other than the operation of the switching button may be an operation of reproducing the dynamic image. After the reproduction of the dynamic image is finished, the display of the display 140 is switched to an image to be displayed next.

In addition, in a case where a dynamic image is included in the analysis result, the dynamic image displayed on the display 140 may be reproduced by a user's operation or may be automatically reproduced.

Alternatively, after all of the analysis results set in the display order are displayed on the display 140, the analysis result displayed first may be displayed on the display 140 again. That is, after all of the plurality of analysis results are displayed on the display 140 in the set display order, the controller 110 may sequentially display the plurality of analysis results on the display 140 again in the display order.

For example, as described above, it is assumed that the display order is set in the order of the original image, the ventilation analysis image, and the adhesion analysis image. In this case, after the original image, the ventilation analysis image, and the adhesion analysis image are sequentially displayed, the original image is displayed on the display 140 again. In addition, in a case where a dynamic image is included in analysis results set in the display order, all frames of the dynamic image may be displayed in the first round, and some frames of the dynamic image may be displayed in the second and subsequent rounds.

The display order set by the controller 110 may be an order according to a diagnosis flow set in advance (i.e., the display order may follow a diagnosis flow set in advance). The diagnosis flow set in advance may be, for example, a diagnosis flow set for each facility.

For example, in a case where there are a plurality of radiologists in a facility, there is a possibility that a difference occurs in a diagnostic result due to a difference in an empirical value of an radiologist in charge. Therefore, by defining a diagnosis flow for each facility, even an inexperienced radiologist can check analysis results in an appropriate display order.

In addition, the diagnosis flow set in advance may be a diagnosis flow set for each medical department.

For example, in a case where the medical department is an emergency department, the urgency is relatively high, and thus it is necessary to check the abnormality detection image at an early timing. On the other hand, even when an abnormality is detected in a medical department other than the emergency department, it is preferable to grasp parts other than the abnormality detection to some extent, and therefore, the checking of the abnormality detection image may be performed at a delayed timing. Further, in the case of monitoring post-operative progress in thoracic surgery or the like, a past comparison image is required.

Therefore, since the diagnosis flow is different depending on the medical department, by defining the diagnosis flow for each medical department, it is possible to display the analysis results on the display 140 in an appropriate display order for each medical department.

In addition, the preset diagnosis flow may be a diagnosis flow set for each user. That is, the controller 110 may set the first display order for the first user and the second display order for the second user as the display order of each of the first user and the second user.

For example, when the user is a radiologist, the user needs to peruse all of the plurality of analysis results. However, in a case where the user is a clinician, although the user intensively views a portion having a problem, the user rarely views all of the plurality of analysis results. Therefore, it is considered that the necessity of the display order similar to that of the radiologist is extremely low.

For these reasons, by defining a diagnosis flow for each user, it is possible to improve the efficiency of checking of analysis results by the user.

In addition, the diagnosis flow for each facility, each medical department, or each user may be set by initial setting, or may be set by changing the diagnosis flow of the initial setting of the apparatus by an editing operation of the user.

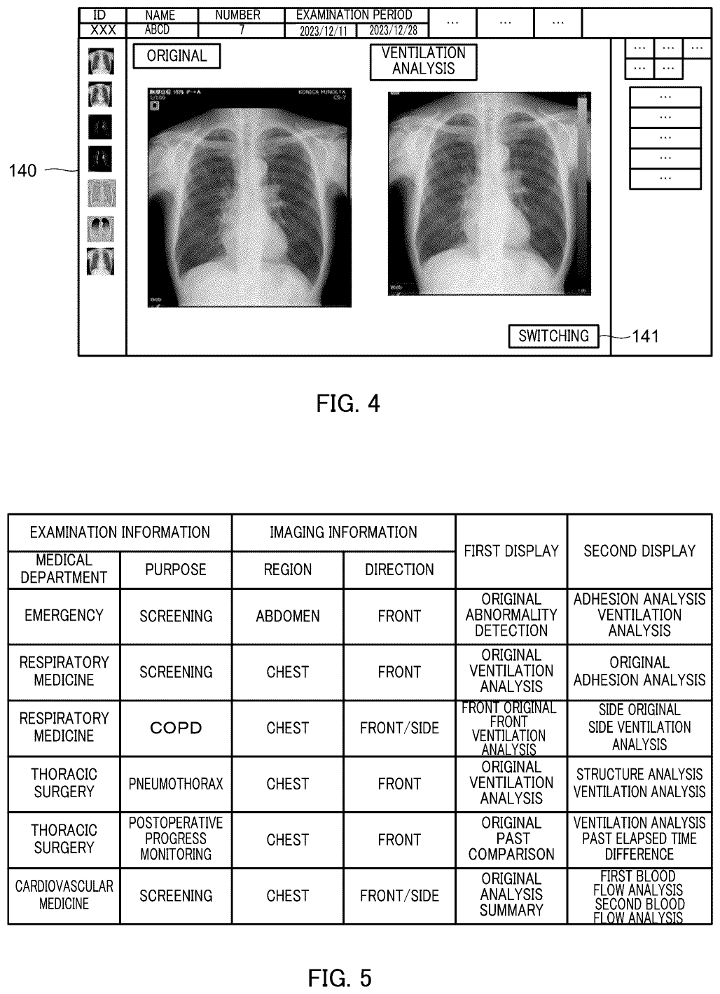

Further, as shown in FIG. 4, the controller 110 may perform control such that a plurality of analysis images, which are based on a plurality of analysis results, are simultaneously displayed on the screen of the display 140. For example, the plurality of analysis images are displayed in A×B display. “A” represents the number of images arranged in the horizontal direction. “B” represents the number of images arranged in the vertical direction.

The controller 110 may set the combination of analysis images to be simultaneously displayed on the screen of the display 140 according to the content of the analysis images.

For example, when an image resulted from performing of analysis processing, such as a ventilation analysis image, is displayed at the same time as an original image, it is easier to compare the images. Therefore, the controller 110 performs control so that the original image and the image on which analysis processing has been performed are displayed in 2×1 display. FIG. 4 illustrates an example in which the original image and the ventilation analysis image are displayed in 2×1 display.

Accordingly, the user can easily check the analysis image. In addition, since the number of times of switching of the analysis result can be reduced, the time for checking the analysis results can be shortened.

In addition, when the analysis images displayed at the same time are, for example, images for different diagnosis purposes such as the ventilation analysis image and the blood flow analysis image, the display of the display 140 becomes complicated and may be difficult for the user to see.

Therefore, the controller 110 may set analysis images that are not displayed at the same time on the screen of the display 140.

As a result, it is possible to prevent the display from being difficult for the user to see.

The controller 110 may also set a plurality of patterns of the display order (plurality of display order patterns) with different combinations of the elements of the examination information and the imaging information and store the display order patterns in the storage 130. FIG. 5 illustrates an example of data in which the plurality of display order patterns are associated with a combination of elements of examination information and imaging information.

The first display in FIG. 5 is a display image to be displayed first. The second display is a display image that is displayed second. Further, FIG. 5 shows an example in which the display order includes only two orders of the first display and the second display, but the display order may include three or more orders.

To be specific, in a case where the medical department is respiratory medicine and the examination purpose is COPD, the region of chest and the direction of front and the side as the imaging information are combined with the elements of the examination information. In the first display, the front original image and the front ventilation analysis image are set in 2×1 display, and in the second display, the side original image and the side ventilation analysis image are set in 2×1 display.

In addition, when the medical department is thoracic surgery and the examination purpose is postoperative progress monitoring, there is a past examination, and the region of the chest and the direction of the front as the imaging information are combined with the elements of the examination information. The original image and the past comparison image are set in the 2×1 display in the first display, and the ventilation analysis image and the past temporal difference image are set in the 2×1 display in the second display.

Thus, a plurality of display patterns can be prepared in advance, so that a plurality of analysis results can be displayed in an appropriate display order on the display 140 at timings at which various users want to check the results.

For example, the controller 110 selects one of the plurality of display patterns stored in the storage 130 based on identification information of a user (for example, a clinician or the like) who checks the display 140. Specifically, the controller 110 selects a corresponding display pattern according to the information on the medical department to which the user belongs.

In this way, a plurality of analysis results can be displayed on the display 140 in an appropriate display order corresponding to the user.

When the user who checks the display 140 is a radiologist, the medical department to which the radiologist belongs may be different from the medical department that has requested the examination of the subject. For example, in a case where the medical department that has requested the examination of the subject is thoracic surgery and the medical department to which the image radiologist belongs is radiology, the medical department to which the image radiologist belongs is different from the medical department that has requested the examination.

In this case, the controller 110 may select one of the plurality of display order patterns based on information associated with the plurality of analysis results.

The information associated with the plurality of analysis results may be, for example, examination information and imaging information.

For example, since the examination information includes information about the medical department that has requested the examination, the controller 110 selects a display pattern corresponding to the medical department from the information about the medical department. Further, in a case where the corresponding medical department is respiratory medicine, when only the information of the front image is included in the imaging direction in the imaging information, the controller 110 selects the display pattern corresponding to only the information of the front image.

In this manner, a plurality of analysis results can be displayed on the display 140 in an appropriate display order corresponding to the analysis results.

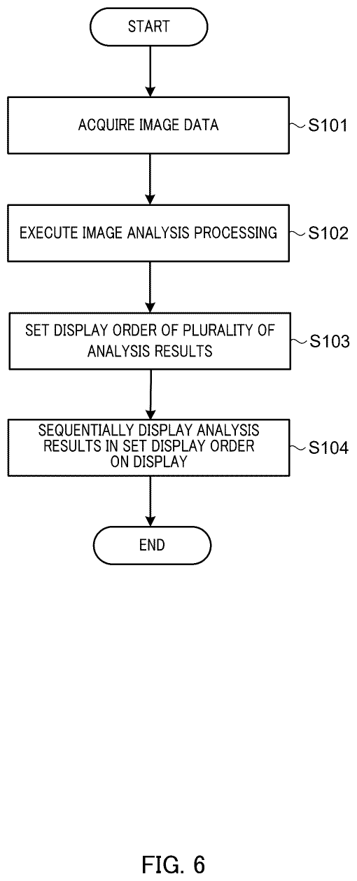

Processing of the controller 110 according to the present embodiment configured as described above will be described. FIG. 6 is a flowchart illustrating an example of processing of the controller 110.

As illustrated in FIG. 6, the controller 110 acquires image data from the modality (step S101). After acquiring the image date, the controller 110 executes image analysis processing on the image data (step S102). After the execution of the image analysis processing, the controller 110 sets the display order of the plurality of analysis results (step S103).

After setting the display order, the controller 110 sequentially displays the plurality of analysis results in the set display order on the display (step S104). After step S104, the present control ends.

According to the present embodiment configured as described above, the display order of the plurality of analysis results for the radiographic image on the display 140 is set, and control is performed such that the plurality of analysis results are sequentially displayed on the display 140 in a plurality of times in accordance with the display order.

Thus, when checking a plurality of analysis results, the user does not have to perform the operation of selecting an analysis result to be displayed from among the plurality of analysis results. In other words, the effort required for the user to check the display of the plurality of analysis results can be reduced.

In addition, since the controller 110 sets the display order so that the display order follows a diagnosis flow set in advance, the user can check the analysis results which need to be checked in an appropriate display order and at a necessary timing.

In addition, for example, since the diagnosis flow can be fixed for a specific case, it is possible to reduce the work load caused by the switching of the display.

In addition, since it is possible to standardize the diagnosis flow including the display order, it is possible to easily share the viewpoint points of the doctor.

In addition, by fixing the diagnosis flow, it is easy to specify an analysis image to be prepared in advance, and thus it is possible to reduce the generation cost of the image.

The controller 110 also performs control such that a plurality of analysis images are simultaneously displayed, which are based on a plurality of analysis results, on the screen of the display 140 and sets, according to the content of the analysis images, a combination of analysis images to be simultaneously displayed on the screen of the display 140.

Accordingly, the user can easily check the analysis image. In addition, since the number of times of switching of the analysis result can be reduced, the time for checking the analysis results can be shortened. In addition, it is possible to suppress a display which is difficult for the user to view due to an analysis image which is not simultaneously displayed on the screen being set according to the content of the analysis image.

In addition, since the controller 110 selects one of the plurality of display order patterns based on the identification information of the checking user who checks the radiographic image, it is possible to cause the display 140 to display the plurality of analysis results in an appropriate display order corresponding to the user.

Furthermore, since the controller 110 selects one of the plurality of display order patterns based on the information associated with the plurality of analysis results, it is possible to cause the display 140 to display the plurality of analysis results in an appropriate display order corresponding to the analysis results.

In the above-described embodiment, the elements of the examination information used for setting the display order are the information of the medical department and the information of the purpose of the examination, but the present invention is not limited thereto. For example, the examination information used for setting the display order may include elements such as information on the sex of the subject, information on the age of the subject, information on the physique of the subject, and information on the history of disease of the subject. Each information of the subject may be information read from an electronic medical record of the subject, information input by the user, or the like.

In this way, since the display order can be set to be different for each subject, the user can check the analysis result in accordance with the display order suitable for the subject.

In the above-described embodiment, the elements of the imaging information used for setting the display order are the information on the examination region of the subject, the information on the presence or absence of the past examination, the information on the imaging direction, and the information on the number of times of imaging, but the present invention is not limited thereto. For example, the imaging information used for setting the display order may include an element relating to the information of the imaging method such as the information of the breathing technique (breath holding, labored breathing, natural breathing, or the like).

In this way, since the analysis images unique to the imaging method can be displayed in accordance with an appropriate display order, it is possible to improve the ease of checking by the user.

Furthermore, although the display order is set according to the elements included in the examination information and the imaging information in the above-described embodiment, the present invention is not limited to this. For example, since the type of analysis processing and the type of machine learning that can be used differ depending on the facility, the display order may be set according to the type of analysis processing and the type of machine learning.

In the above-described embodiment, the display order is set in accordance with a predetermined flow regardless of the result of the analysis processing or the like, but the present invention is not limited thereto. For example, the controller 110 may change the display order according to a predetermined flow in accordance with the result of analysis processing, the result of case estimation by CAD processing, or the like.

Specifically, a display object to be simultaneously displayed on the display 140 may be added or changed. In addition, in a case where analysis and determination are performed by machine learning, a display order may be set such that an image to be displayed next can be selected by the user according to an analysis result.

Thus, since the display order reflects the result of analysis processing and the like, it is possible to further improve the ease of checking by the user.

In addition, in a case where the analysis image is a dynamic image, the controller 110 may perform control such that an initial display frame of the dynamic image is selected and displayed on the display 140 based on the analysis content. Specifically, for example, a frame in which an abnormality is detected, a frame having the maximum exhalation amount or the maximum inhalation amount in a dynamic image in which breathing is performed, a frame serving as a starting point of the movement of the subject in a dynamic image for the purpose of shaping, or the like may be selected as the initial display frame.

Thus, since the frame to be checked in the dynamic image is initially displayed, the user can quickly check the important frame without fully reproducing the dynamic image.

Furthermore, the controller 110 may use the first frame of the dynamic image as the initial display frame, regardless of the analysis content.

Thus, the user can check the dynamic image thoroughly.

Incidentally, it is assumed that the user discovers an important frame to be checked (hereinafter, important frame) during the checking of the predetermined dynamic image. In this case, since the user is aware of the position of the important frame in the dynamic image displayed next to the predetermined dynamic image, from the viewpoint of shortening the checking time, it is considered to be preferable that the frame corresponding to the important frame of the predetermined dynamic image is displayed as quickly as possible.

Therefore, in a case where the user performs setting to add an annotation to a predetermined frame in a first dynamic image among the plurality of dynamic images, the controller 110 may change the initial display frame of a second dynamic image (which is after the first dynamic image in the display order) to be set to a frame related to the predetermined frame.

The setting to add an annotation is a setting operation performed by the user via the operation unit or the like, such as adding a mark to a specific frame in the dynamic image. The setting may be any setting operation as long as the controller 110 can identify the specific frame. The specific frame may be an image in which a case is most easily diagnosed among all the frames or an image in which a characteristic point such as adhesion is displayed. The setting operation by which the controller 110 can identify the specific frame may include, in addition to the marking operation described above, an operation of adding an arrow, an operation of enclosing with a rectangle, an operation of adding a comment to the specific frame, and the like.

The frame related to the predetermined frame may be a frame corresponding to the same place as the predetermined frame of the first dynamic image in the second dynamic image or a frame several frames before the frame corresponding to the same place as the predetermined frame.

For example, it is assumed that, during reproduction of the first dynamic image, the user has performed setting to add an annotation to the tenth frame because the tenth frame is considered to be an important frame. In this case, when the second dynamic image is reproduced, the initial display frame is set to, for example, the tenth frame.

As a result, it is possible to shorten the checking time of the dynamic image as a whole.

Incidentally, in a case where an analysis result is checked by a plurality of persons, it is assumed that a diagnosis flow is set in units of users, and at this time, a user (first user) who first checked the analysis result has found an abnormality in a predetermined analysis image. In this case, when the display order of the analysis image in which the abnormality is detected is set to the later order (set to be displayed later) in advance, it is inconvenient for a user (second user) who checks the analysis result later because the analysis image which the user wants to quickly check is displayed later.

Therefore, the controller 110 may change the display order set for the second user from the second display order based on the checking information of the plurality of analysis results based on the first display order by the first user. The first display order is a display order set for the first user, and the second display order is a display order set for the second user.

For example, it is assumed that, in a predetermined facility, a first radiologist first checks an analysis result and then a second radiologist checks the analysis result. Assume that the display order for the first radiologist is set for the first radiologist, and the display order for the second radiologist is set for the second radiologist. It is assumed that the plurality of analysis results include a first analysis result, a second analysis result, a third analysis result, and a fourth analysis result. The first display order is assumed to be a display order in which the first analysis result, the second analysis result, the third analysis result, and the fourth analysis result are displayed on the display 140 in this order. The second display order is assumed to be a display order in which the second analysis result, the third analysis result, and the fourth analysis result are displayed on the display 140 in this order.

In this case, it is assumed that the first radiologist has determined that an abnormality has been detected in the third analysis result. At this time, the first radiologist inputs the third analysis result as checking information to the image analysis apparatus 15 or the like via the operation unit. When the second user checks the analysis results, the controller 110 changes the display order set for the second user from the second display order to a display order, starting from the third analysis result, the second analysis result, and to the fourth analysis result, based on the checking information.

Accordingly, it is possible to flexibly change the display order for the second user to the display order in which the checking result of the first user checked earlier is reflected.

While the plurality of analysis images are displayed side by side on the screen of the display 140 in the aforementioned embodiment, the present invention is not limited to this. For example, a plurality of analysis images may be displayed on the display 140 in a superimposed manner.

For example, in the blood flow analysis, in order to check the state of the left lung of the subject, the two analysis images may be superimposed and displayed so that the states of the respective left halves of the two kinds of blood flow analysis images are aligned. In addition, analysis images whose analysis results do not interfere with each other (for example, each image of the structure analysis and the ventilation analysis) may be superimposed and displayed as layers.

Furthermore, although the display order is set in the above embodiment, when the user is not aware of the display order, the user might not be able to understand the analysis result to be displayed next when the analysis result is switched, but the present invention is not limited to this. For example, the controller 110 may display the information of the second analysis image to be displayed next to the currently displayed first analysis image on the display 140 simultaneously with the first analysis image.

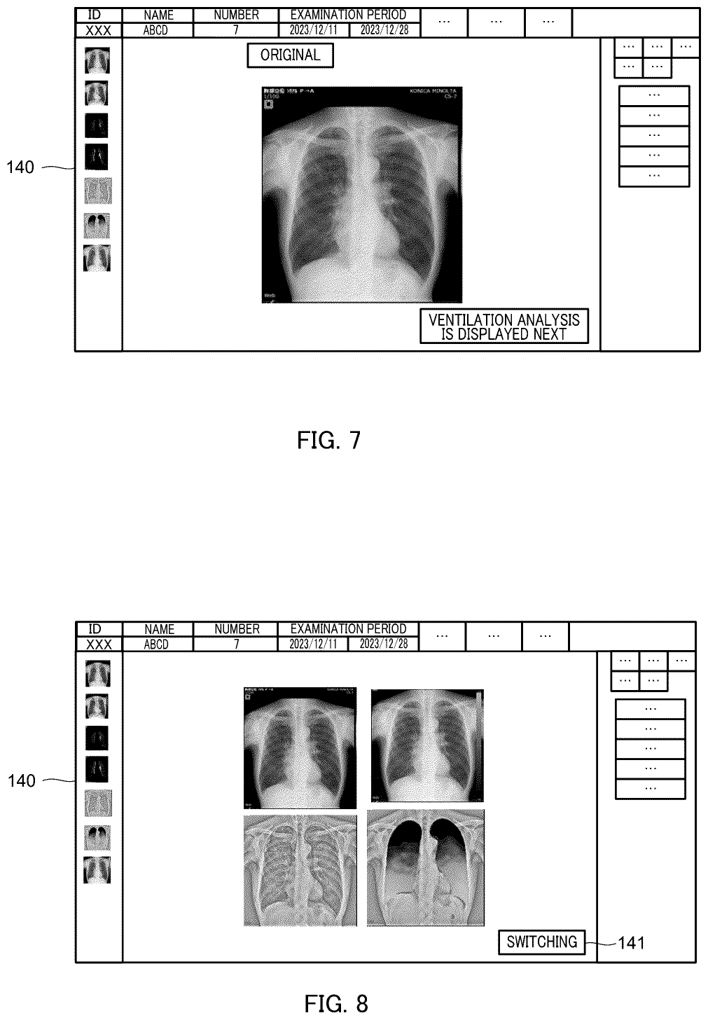

For example, FIG. 7 illustrates an example in which the information on the second analysis image is displayed below the first analysis image (original image) on the display screen. In FIG. 7, information indicating that the next image to be displayed is a “ventilation analysis” is shown as an example of information on the second analysis image.

Furthermore, although the 2×1 display is given as an example in which a plurality of analysis images are displayed at the same time in the embodiment described above, the present invention is not limited to this example, and any display other than the 2×1 display, such as the 2×2 display (refer to FIG. 8), may be used, for example.

In addition, in the above-described embodiment, the image analysis apparatus 15 is exemplified as the display control apparatus, but the invention is not limited thereto, and for example, another device such as a PACS14 may be the display control apparatus.

Furthermore, although the display control apparatus is a device (image analysis apparatus) having a display in the above-described embodiment, the present invention is not limited to this, and the display control apparatus may be a device having no display. This device need only be configured to acquire analysis results and the like analyzed by the image analysis apparatus, set the display order, and display the analysis results in the set display order on another display device.

In addition, each of the above-described embodiments is merely an example for embodying the present invention, and the technical scope of the present invention should not be interpreted in a limited manner by these embodiments. That is, the present invention can be implemented in various forms without departing from the spirit or main features thereof.

Although embodiments of the present invention have been described and illustrated in detail, the disclosed embodiments are made for purposes of illustration and example only and not limitation. The scope of the present invention should be interpreted by terms of the appended claims.

What is claimed is:

1. A display control apparatus, comprising:
a hardware processor that sets a display order of a plurality of analysis results for a radiographic image to be displayed on a display, wherein
the hardware processor performs control such that the plurality of analysis results are sequentially displayed on the display in a plurality of times in accordance with the set display order.

2. The display control apparatus according to claim 1, wherein
the hardware processor sets the display order such that the display order follows a diagnosis flow set in advance.

3. The display control apparatus according to claim 1, wherein
the hardware processor performs control such that a plurality of analysis images are simultaneously displayed on a screen of the display, the plurality of analysis images being based on the plurality of analysis results; and
the hardware processor sets, according to content of the analysis images, a combination of analysis images to be simultaneously displayed on the screen of the display.

4. The display control apparatus according to claim 1, wherein:
the plurality of analysis results include a dynamic image; and
the hardware processor performs control such that an initial display frame of the dynamic image is selected and displayed on the display based on analysis content in the plurality of analysis results.

5. The display control apparatus according to claim 4, wherein:
the plurality of analysis results include a plurality of the dynamic images; and
when a user performs setting to add an annotation to a predetermined frame in a first dynamic image among the plurality of dynamic images, the hardware processor changes an initial display frame of a second dynamic image to be set to a frame related to the predetermined frame, the second dynamic image being an image after the first dynamic image in the display order.

6. The display control apparatus according to claim 1, wherein:
the hardware processor sets a first display order for a first user as the display order for the first user and sets a second display order for a second user as the display order for the second user; and
the hardware processor changes the display order set for the second user from the second display order, based on checking information of the plurality of analysis results by the first user based on the first display order.

7. The display control apparatus according to claim 1, wherein:
the hardware processor sets a plurality of patterns of the display order in the plurality of analysis results, based on a combination of an element included in a plurality of types of examination information and an element included in a plurality of types of imaging information; and
the hardware processor selects one of the plurality of patterns of the display order based on identification information of a checking user who checks the radiographic image.

8. The display control apparatus according to claim 1, wherein:
the hardware processor sets a plurality of patterns of the display order in the plurality of analysis results, based on a combination of an element included in a plurality of types of examination information and an element included in a plurality of types of imaging information; and
the hardware processor selects one of the plurality of patterns of the display order based on information associated with the plurality of analysis results.

9. A method for controlling display of a plurality of analysis results for a radiographic image, the method comprising:
setting a display order of the plurality of analysis results on a display; and
performing control such that the plurality of analysis results are sequentially displayed on the display in a plurality of times in accordance with the set display order.

10. A non-transitory computer-readable recording medium storing a display control program for a plurality of analysis results for a radiographic image, the display control program causing a computer to execute:
setting a display order of the plurality of analysis results to be displayed on a display; and
performing control such that the plurality of analysis results are sequentially displayed on the display in a plurality of times in accordance with the set display order.
